SEQUENCE LISTING (11 sequences)

SEQ ID NO: 1 - protein, 957 aa, Homo sapiens
MNLQDIRYILKNDLKDFTGAQRTQPTESPRVQRYGLHPDVNVYLGLTSEHPRETPDMEIIELKEMGSQPHSPRVHSLFTEGTLDPQAPDPCLMARETQNQDAPCPAPFMAEEASSPSTGQPSLCSFEINEIYSGCLILEDDIEEPPGAASSLEADGPNQVDELKSMEEELDKMEREACCFGSEDESSSKAETEYSFDDWDWQNGSLSSLSLPESTREAKSNLNNMSTTEEYLISKCVLDLKIMQTIMHENDDRLRNIEQILDEVEMKQKEQEERMSLWATSREFTNAYKLPLAVGPPSLNYIPPVLQLSGGQKPDTSGNYPTLPRFPRMLPTLCDPGKQNTDEQFQCTQGAKDSLETSRIQNTSSQGRPRESTAQAKATQFNSALFTLSSHRQGPSASPSCHWDSTRMSVEPVSSEIYNAESRNKDDGKVHLKWKMEVKEMAKKAATGQLTVPPWHPQSSLTLESEAENEPDALLQPPIRSPENTDWQRVIEYHRENDEPRGNGKFDKTGNNDCDSDQHGRQPRLGSFTSIRHPSPRQKEQPEHSEAFQASSDTLVAVEKSYSTSSPIEEDFEGIQGAFAQPQVSGEEKFQMRKILGKNAEILPRSQFQPVRSTEDEQEETSKESPKELKEKDISLTDIQDLSSISYEPDSSFKEASCKTPKINHAPTSVSTPLSPGSVSSAASQYKDCLESITFQVKTEFASCWNSQEFIQTLSDDFISVRERAKKLDSLLTSSETPPSRLTGLKRLSSFIGAGSPSLVKACDSSPPHATQRRSLPKVEAFSQHRIDELPPPSQELLDDIELLKQQQGSSTVLHENTASDGGGTANDQRHLEEQETDSKKEDSSMLLSKETEDLGEDTERAHSTLDEDLERWLQPPEESVELQDLPKGSERETNIKDQKVGEEKRKREDSITPERRKSEGVLGTSEEDELKSCFWKRLGWSESSRIIVLDQSDLSD

SEQ ID NO: 2 - protein, 7 aa, Artificial Sequence
  Synthetic peptide
GPPXXXY

SEQ ID NO: 3 - protein, 7 aa, Artificial Sequence
  Synthetic peptide
AAAXXXA

SEQ ID NO: 4 - DNA, 24 bp, Artificial Sequence
  Synthetic primer
cgttggtcagcccagaagtcatca

SEQ ID NO: 5 - DNA, 25 bp, Artificial Sequence
  Synthetic primer
gggcccagcagtatggtttcctaca

SEQ ID NO: 6 - DNA, 22 bp, Artificial Sequence
  Synthetic primer
cttttctgggaaccagtggagt

SEQ ID NO: 7 - DNA, 22 bp, Artificial Sequence
  Synthetic primer
ttatggccagaaatcactgcat

SEQ ID NO: 8 - DNA, 22 bp, Artificial Sequence
  Synthetic primer
acagcgatggttagggtctgag

SEQ ID NO: 9 - DNA, 22 bp, Artificial Sequence
  Synthetic primer
ggacctagaccacgagcattta

SEQ ID NO: 10 - DNA, 22 bp, Artificial Sequence
  Synthetic primer
cgctgttaggccttggtaagat

SEQ ID NO: 11 - DNA, 22 bp, Artificial Sequence
  Synthetic primer
tcttctttgcttccacttgctg

(19) United States
(12) Patent Application Publication
(10) Pub. No.: US 2013/0225503 A1
(43) Pub. Date: Aug. 29, 2013

(54) TEX14 PEPTIDES AS NOVEL ANTITUMOR AGENTS

(71) Applicants: Martin M. Matzuk, Houston, TX (US); Tokuko Iwamori, Houston, TX (US); Michael P. Greenbaum, Nashville, TN (US); Lang Ma, Houston, TX (US); Naoki Iwamori, Houston, TX (US)

(72) Inventors: Martin M. Matzuk, Houston, TX (US); Tokuko Iwamori, Houston, TX (US); Michael P. Greenbaum, Nashville, TN (US); Lang Ma, Houston, TX (US); Naoki Iwamori, Houston, TX (US)

(73) Assignee: Baylor Licnensing Group, Houston, TX (US)

(21) Appl. No.: 13/499,401

(22) PCT Filed: Sep. 30, 2010
(86) PCT No.: PCT/US10/50842

§ 371 Date: Nov. 29, 2012

Related U.S. Application Data
(60) Provisional application No. 61/247,332, filed on Sep. 30, 2009.

(51) Int. Cl. C07K 14/47; A61K 38/17; A61K 45/06

(52) U.S. Cl. CPC C07K 14/47; A61K 45/06; A61K 38/1709
USPC 514/19.4; 530/329; 530/350; 514/19.3; 514/19.5; 435/375; 600/1

(57) ABSTRACT

Embodiments of the present invention regard TEX14 peptides for cancer treatment. In particular, the TEX14 peptides comprise a GPPX3Y motif. Methods, compositions, and kits are encompassed.

### CROSS-REFERENCE TO RELATED APPLICATIONS

The present invention claims priority to U.S. Provisional Patent Application Ser. No. 61/247,332, filed Sep. 30, 2010, which is incorporated by reference herein in its entirety.

### STATEMENT REGARDING FEDERALLY SPONSORED RESEARCH OR DEVELOPMENT

This invention was made with government support under R01HD057880 and U01HD060496 awarded by the National Institutes of Health. The United States Government has certain rights in the invention.

### TECHNICAL FIELD

The present invention generally concerns the fields of cell biology, molecular biology, and medicine, including cancer medicine.

### BACKGROUND OF THE INVENTION

Cytokinesis is the process by which a single cell separates into two genetically identical daughter cells (8). The events of cytokinesis begin shortly after the sister chromatids separate during mitotic anaphase. As the contractile ring narrows, the future daughter cells are connected by a narrow channel in which the evolutionarily conserved centralspindlin complex (a heterotetrameric complex of MKLP1 and MgcRacGap) localizes (16). Centrosomal 55-kDa protein (CEP55) is recruited from the centrosome to this centrally located complex and interacts with MKLP1 (6, 15, 22). Subsequently, ALIX (ALG-2 interacting protein X, also known as programmed cell death 6 interacting protein) and TSG101 (a component of the ESCRT-1 [endosomal sorting complex required for transport-1] complex) are recruited to the midbody through coiled-coil interactions with the CEP55 homodimer (3, 15, 17). Glycine (G)-proline (P)-proline (P)—X—XX-tyrosine (Y) (GPPX3Y) motifs in ALIX and TSG101 are critical for this interaction with CEP55 (14, 17). Knockdown experiments in somatic cells have revealed that a deficiency of CEP55 leads to incomplete abscission and formation of multinucleated cells (3, 17). In addition, knockdown of either TSG101 or ALIX, known direct downstream interacting partners of CEP55, leads to a similar phenotype (3, 17). These interactions are essential for somatic cell abscission (2, 3, 17).

In contrast to these abscission events in somatic cells, differentiating germ cells do not complete cytokinesis and instead are linked together through 0.5- to 3-□m electron-dense “channels” called intercellular bridges (5, 7, 12). Intercellular bridges are evolutionarily conserved structures that are present in the gonads of essentially all multicellular organisms from fruit flies and hydra to marsupials, mice, and humans. In mammals, intercellular bridges play roles in synchronization of germ cells by passage of organelles and molecules between germ cells (especially important postmeiotically in haploid spermatids) (1, 19).

It has been previously shown that testis expressed gene 14 (TEX14) localizes to male and female germ cell intercellular bridges (9, 11) and that the bridge forms through a direct interaction between TEX14 and the MKLP1-containing midbody protein complex (10). TEX14-positive intercellular bridges interconnect human and mouse spermatogonia as soon as spermatogonia begin to differentiate and continue to interconnect male germ cells up through formation of mature spermatozoa (11). Targeted deletion of TEX14 disrupts intercellular bridges in germ cells and causes sterility in male mice (11) but not in female mice (9). Furthermore, not only do MKLP1 and TEX14 interact in male germ cells, but MKLP1 and its centralspindlin complex partner, MgcRacGap, become stable components of the intercellular bridge (10). These results demonstrate that intercellular bridges are essential for spermatogenesis; however, until now, it was unclear how TEX14 participated in intercellular bridge formation to prevent abscission and the completion of cytokinesis in male germ cells. We demonstrate here that a TEX14-CEP55 interaction is critical for subverting abscission toward a stable intercellular bridge.

### BRIEF SUMMARY OF THE INVENTION

In somatic cells, abscission, the physical separation of daughter cells at the completion of cytokinesis, requires CEP55, ALIX, and TSG101. In contrast, cytokinesis is arrested prior to abscission in differentiating male germ cells that are interconnected by TEX14-positive intercellular bridges. Targeted deletion of TEX14 disrupts intercellular bridges in all germ cells and causes male sterility.

Although these findings demonstrate that intercellular bridges are essential for spermatogenesis, prior to the present disclosure it was unknown how TEX14 and other proteins come together to prevent abscission and form stable intercellular bridges. Using a biochemical enrichment of male germ cell intercellular bridges, additional bridge proteins were identified, including CEP55. Although CEP55 is highly expressed in testes at the RNA level, there is no report of the presence of CEP55 in germ cells. As shown herein, CEP55 becomes a stable component of the intercellular bridge, and an evolutionarily conserved GPPX3Y motif of TEX14 binds strongly to CEP55 to block similar GPPX3Y motifs of ALIX and TSG101 from interacting and localizing to the midbody. Thus, TEX14 prevents the completion of cytokinesis by altering the destiny of CEP55 from a nidus for abscission to an integral component of the intercellular bridge.

In certain embodiments of the invention, this aspect of the GPPX3Y of TEX14 is exploited for methods and compositions for the treatment and/or prevention of cancer. In particular, peptides of TEX14 that include the GPPX3Y motif are utilized to target cancer cells to prevent their proliferation.

In one embodiment of the invention, there is a composition comprising a TEX14 peptide, said peptide having sequence comprising SEQ ID NO:2. In a specific embodiment, the TEX14 peptide is not more than 90, 85, 80, 75, 70, 65, 60, 55, 50, 45, 40, 35, 30, 25, 20, 19, 18, 17, 16, 15, 14, 13, 12, 11, 10, 9, 8, or 7 amino acids in length. In a certain case, the peptide sequence other than SEQ ID NO:2 of the TEX14 peptide comprises the corresponding peptide sequence of SEQ ID NO:1. In some aspects, the peptide sequence other than SEQ ID NO:2 of the TEX14 peptide is at least 75%, 80%, 85%, 90%, 95%, 97%, or 99% identical to the corresponding peptide sequence of SEQ ID NO:1. In certain cases, the composition is further defined as including a pharmaceutical carrier.

In some embodiments, there is a method for inhibiting the proliferation of a mammalian cell, comprising contacting the cell with a TEX14 peptide. In specific embodiments, the mammalian cell is a cancer cell of an individual, such as a cancer cell of the lung, breast, prostate, pancreas, brain, blood, liver, colon, gall bladder, pituitary gland, spleen, esophagus, ovary, testis, cervix, kidney, salivary gland, anus, skin or thyroid. In a specific embodiment, the method further comprises administering an additional cancer therapy to the individual, such as surgery, radiation, chemotherapy, immunotherapy, or hormone therapy, for example.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### BRIEF DESCRIPTION OF THE DRAWINGS

FIGS. 1A-1B concern CEP55 as a component of the intracellular bridge. (A) Western blot analysis of intercellular bridge enrichment using the anti-TEX14 and anti-CEP55 antibodies. (C) The 24 CEP55 peptides that were identified by proteomic analysis. The identified peptides are highlighted in yellow and green, with shorter overlapping peptides underlined.

FIGS. 2A-2C show that TEX14 interacts with CEP55. (A) Immunoprecipitation (IP) of FLAG-TEX14 and/or MYC-CEP55 from HEK293T cells, followed by Western blot analysis with the antibodies as shown. Immunoprecipitation of protein G (lanes G) is the control. (B) Yeast two-hybrid analyses using vectors encoding full-length mouse TEX14, MKLP1, CEP55, and positive and negative controls. The relative ratios of TEX14-TEX14, TEX14-MKLP1, and TEX14-CEP55 interactions were determined by using an oxygen-biosensor system. (C) Yeast two-hybrid interactions of mouse and human full-length TEX14 and CEP55. (a) Yeast were stably transformed with vectors and plated for yeast two-hybrid analysis as depicted. SV40 T antigen with p53 and SV40 T antigen with lamin C were used as positive and negative controls, respectively. (b) Interactions between the two-hybrid proteins are evident by colony growth and blue color on selection plates. (c) A non-selection plate shows that all of the transformed yeast are capable of growing.

FIG. 3 illustrates the region encoding the conserved TEX14 GPPX3Y motif interacts with the hinge region of CEP55. (A) The full-length and truncated regions of mouse TEX14 and CEP55 were cloned into the yeast two-hybrid vectors and used for the studies in panels B and C below. (B and C) Yeast two-hybrid oxygen biosensor between the full-length and truncated TEX14 proteins and the full-length CEP55 protein and/or selection plate analyses of the full-length and truncated TEX14 and CEP55 proteins. The terms “+” and “−” indicate positive and negative interactions, respectively. (D) Alignment of the GPPX3Y motif and flanking sequences from TEX14 orthologs and human ALIX and TSG101. Conserved amino acids are highlighted.

FIG. 4 shows essential motifs (A) and sequences (B) of TEX14, CEP55, ALIX, and TSG101. (A) The sizes and the domains/motif-containing regions of full-length TEX14, CEP55, ALIX, and TSG101 are shown. The domain regions highlighted in red are referred to as TX, CEP, ALIX, and TSG. (B) The corresponding amino acid sequences of these regions with the conserved consensus sequences in mouse and human are shown. These truncated proteins were used for the mammalian two-hybrid assays.

FIGS. 5A-5F show that the GPPX3Y motif of TEX14 is essential for binding to the hinge region of CEP55. (A and B) Summary of the modified mammalian two-hybrid assays (A) Three kinds of transfection vectors were made. One protein coding sequence (“X”) was fused to a transcriptional activation domain sequence (VP16-AD), and the other protein coding sequence (“Y”) was fused to a DNA-binding domain sequence (GAL4-BD). (B) When proteins “X” and “Y” interact, transcriptional activation of the mCherry gene occurs, which is detected as red fluorescence (B, top right). The GAL4-BD-Y vector expresses the Renilla reniformis luciferase, allowing for normalization of transfections. The relative interaction of protein X and Y is determined by the mCherry/Renilla reniformis luciferase ratio. (C to F) Mammalian-two-hybrid interactions of chimeric VP16-AD-X and GAL4-BD-Y proteins in transfected HEK293T cells is shown (see FIG. 4 for additional details).

FIGS. 6A-6G show that the GPPX3Y motif of TEX14 inhibits the CEP55-ALIX and CEP55-TSG101 interactions and the entry of ALIX to the midbody, resulting in formation of stable intercellular bridges. (A to C) pcDNA3 vectors lacking an insert (Empty) or containing the truncated TEX14 (TX), ALIX (ALIX), TSG101(TSG), and TEX14 mutant (AAAX3A) were cotransfected into HEK293T cells along with VP16-AD-X, GAL4-BD-Y, and GAL4.31-mCherry vectors indicated at the bottom of each panel (see FIG. 4B for additional details). The relative ratios of the interactions of protein X and protein Y are shown. (D) Transfection of the full-length TEX14 vectors into HeLa cells. Immunofluorescence using goat anti-TEX14 and guinea pig anti-MKLP1 antibodies was performed: red, TEX14; green, MKLP1; blue, DAPI; and yellow, merged. (E and F) Cotransfection of pcDNA-YFP-full-length ALIX overexpression vector with pcDNA-mCherry-truncated GPPX3Y TEX14 (TX) (E) and TEX14 mutant AAAX3A (F) overexpression vectors into HeLa cells. The localization patterns of ALIX were microscopically examined for yellow fluorescence within a background of cells expressing TX or AAAX3A (red fluorescence). Arrow, midbody; arrowhead, ALIX. (G) Quantification of the experiment in panels E and F. The graphs were made by analyzing 1,000 double-positive cells with RFP and YFP from 11 to 13 separate experiments. The graphs show the percentage of the number of bridge containing cells/the number of double-positive RFP and YFP cells (left) and the number of ALIX localized in midbody/the number of RFP positive bridges in double-positive RFP and YFP cells (right).

FIG. 7 demonstrates that the GPPX3Y-containing TEX14 region interacts with CEP55 much more strongly than the equivalent regions of ALIX and TSG101 and inhibits the CEP55-ALIX and CEP55-TSG101 interactions. The pcDNA3 vectors lacking an insert (Empty) or containing the truncated TEX14 (TX), ALIX (ALIX), TSG101(TSG), and TEX14 mutant (AAAX3A) were cotransfected into HEK293T cells, along with VP16-AD-X, GAL4-BD-Y, and GAL4.3.1-mCherry vectors indicated at the bottom of each panel. The GAL4-BD-Y vector, containing the Renilla luciferase sequence, was replaced by yellow fluorescent protein (YFP) sequence. YFP expression was used for normalization of transfections instead of Renilla luciferase. The relative interaction of proteins X and Y is determined by the mCherry/YFP ratio. The interactions of chimeric VP16-AD-X and GAL4-BD-Y proteins in transfected HEK293T cells are shown (see FIG. 4B for additional details).

FIG. 8. Illustrates exemplary models for cytokinesis and intercellular bridge formation. (Left) Model of somatic cell abscission. CEP55 is essential in the recruitment of additional proteins (e.g., TSG101 and ALIX) that are required for abscission of the midbody (2, 3, 17). The regions of TSG101 and ALIX containing GPPX3Y motifs interact with the hinge region of CEP55 (14, 17) to complete cytokinesis. (Right) Exemplary model of the intercellular bridge in differentiating germ cells. The conserved GPPX3Y motif of TEX14 interacts strongly with the hinge region of CEP55 in differentiating germ cells to block CEP55 interactions with TSG101 and ALIX, resulting in formation of a stable intercellular bridge.

FIG. 9 illustrates exemplary TEX14 peptides and the effect of HeLa cell growth.

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

### I. Exemplary Peptides of the Invention

In certain embodiments of the invention, the methods and compositions concern the motif GPPX3Y in a peptide. The peptide may be used for any purpose, although in specific embodiments the peptide is employed for cancer treatment. The cancer treatment may be for any kind of cancer, but in specific embodiments the cancer is lung, brain, breast, liver, pancreatic, bone, blood, spleen, colon, ovarian, testicular, cervical, gall bladder, esophageal, anal, bladder, or kidney cancer.

The peptide may be formulated in a pharmaceutical composition. In certain embodiments, the peptide comprises the GPPX3Y motif. The peptide may be of any length, but in specific embodiments the peptide is no longer than 200 amino acids, 150 amino acids, 100 amino acids, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 34 amino acids, 33 amino acids, 32 amino acids, 31 amino acids, 30 amino acids, 29 amino acids, 28 amino acids, 27 amino acids, 26 amino acids, 25 amino acids, 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, or 7 amino acids. In certain embodiments, the peptide comprises a length of from 30-50, 30-40, 30-35, 40-50, 45-50, 25-50, 25-45, 25-40, 25-35, 25-30, 20-50, 20-45, 20-40, 20-35, 20-30, 20-25, 15-50, 15-45, 15-40, 15-35, 15-30, 15-25, 15-20, 10-50, 10-45, 10-40, 10-35, 10-30, 10-25, 10-20, 7-50, 7-45, 7-40, 7-35, 7-30, 7-25, 7-20, 7-19, 7-18, 7-17, 7-16, 7-15, 7-14, 7-13, 7-12, 7-11, or 7-10 amino acids. In specific cases, the peptide length is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more amino acids in length.

In specific embodiments, the sequence of the peptide outside of the GPPX3Y (SEQ ID NO:2) motif in the peptide is the underlying human TEX14 sequence. SEQ ID NO:1 provides the human TEX14 as a reference sequence. In specific embodiments, the sequence of the peptide has 1, 2, 3, 4, 5, 6, 7, 8, 9, 10, or more variations compared to the underlying TEX14 sequence. In certain cases, the sequence of the peptide outside of the GPPX3Y motif is 70%, 75%, 80%, 85%, 90%, 91%, 92%, 93%, 94%, 95%, 96%, 97%, 98%, or 99% identical to the TEX14 sequence.

In some cases, the GPPX3Y motif has one, two, or three modifications to it, such as amino acid changes, including conservative amino acid changes, for example.

### II. Pharmaceutical Preparations

Pharmaceutical compositions of the present invention comprise an effective amount of one or more TEX14 peptides dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one TEX14 peptide will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The TEX14 peptide may be contained in different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The TEX14 may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include TEX14 and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the TEX14 peptide may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes. In specific embodiments, the TEX14 peptide is administered to an individual in a liposome.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the TEX14 peptides are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, TEX14 peptides may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound TEX14 peptide may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### III. Combination Therapy

In some embodiments, the present invention is administered to an individual when the individual has been or is currently being treated with another cancer treatment or will be treated with another cancer treatment, or a combination thereof.

Thus, in some embodiments, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

It is contemplated that one or more TEX14 peptides could be used similarly in conjunction with chemotherapeutic, radiotherapeutic, or immunotherapeutic intervention, in addition to other pro-apoptotic or cell cycle regulating agents. Alternatively, the TEX14 peptides may precede or follow the other agent treatment by intervals ranging from minutes to weeks. In embodiments where the other agent and expression construct are applied separately to the cell, one would generally ensure that a significant period of time did not expire between the time of each delivery, such that the agent and TEX14 would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, TEX14 therapy is “A” and the secondary agent, such as radio- or chemotherapy, is “B”:

A/B/A B/A/B B/B/A A/A/B A/B/B B/A/A A/B/B/B B/A/B/B

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with TEX14 therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a secondary gene therapy in which a second therapeutic polynucleotide is administered before, after, or at the same time as the TEX14 therapy. Delivery of a vector encoding one of the following gene products will have an anti-hyperproliferative effect on target tissues. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl 2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl 2 cell death regulatory proteins which share in common structural and sequence homologies. These different family members have been shown to either possess similar functions to Bcl 2 (e.g., BclXL, BclW, BclS, Mcl-1, A1, Bfl-1) or counteract Bcl 2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### IV. Kits of the Invention

Any of the compositions described herein may be comprised in a kit. In a non-limiting example, a compound utilized in detection of telomerase activity is comprised in a kit. The kits will thus comprise, in suitable container means, at least one reagent utilized in embodiments of the present invention.

The kits may comprise a suitably aliquoted composition of the invention, including a TEX14 peptide. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the compositions in close confinement for commercial sale. Such containers may include injection or blow molded plastic containers into which the desired vials are retained, for example.

In certain embodiments of the invention, a sodium chloride solution, ammonium sulfate solution, dialysis buffer, or components thereof (for example, magnesium chloride, Tris HCl, NaOH) are provided. Centrifuge tubes may be provided in the kit, in certain embodiments. In specific embodiments, reagents or devices utilized in blood collecting are provided, such as a needle and vial. Other devices may be included, for example, a means to collect a biopsy sample, may be provided, such as a scalpel, needle, and so forth.

### EXAMPLES

The following examples are offered by way of example and are not intended to limit the scope of the invention in any manner.

### Example 1

### Exemplary Materials and Methods

Enrichment of Intercellular Bridges.

Intercellular bridge preparations were obtained from an 8-week-old wild-type mouse testis as previously described (10). The enriched intercellular bridge fractions were used for Western blot assays, and the final fraction PT was transferred to Superfrost/Plus microscope slides (Fisher Scientific) and allowed to air dry. After drying, the slides were lightly rinsed in TBS (100 mM Tris-HCl [pH 7.5]; 0.9%/150 mM NaCl) and used for immunofluorescence detection of mouse CEP55 and TEX14, as described below.

Production of Anti-CEP55 and Anti-MKLP1 Antibodies.

Antibodies to fulllength mouse CEP55 and MKLP1 protein were generated in guinea pigs using methods described previously (11). The antibodies were purified with the CEP55 or MKLP1 antigens, respectively, using the ProFound mammalian coimmunoprecipitation kit (Pierce).

Immunofluorescence Analysis.

Mouse testes and ovaries were fixed overnight at 4° C., and cultured cells were fixed for 10 min at room temperature in 4% paraformaldehyde in TBS, followed by three washes in 70% ethanol and then overnight at 4° C. in 70% ethanol. The testes and ovaries were processed and embedded by the Department of Pathology Core Services Laboratory (Baylor College of Medicine), and 4-μm sections were cut and prepared for immunostaining.

Samples were blocked in 3 or 5% bovine serum albumin (BSA)-TBS blocking buffer for 1 h at room temperature. Antibodies were diluted in 3 or 5% BSA-TBS blocking buffer and used for overnight incubation at 4° C. at the following dilutions: rabbit or goat anti-TEX14, 1:500; guinea pig anti-CEP55, 1:500; and guinea pig anti-MKLP1, 1:200. Alexa 488- and Alexa 594-conjugated secondary antibodies were purchased from Molecular Probes. Samples were mounted with VectaShield mounting medium with DAPI (4′,6′-diamidino-2-phenylindole; Vector), covered with microscope coverslips (VWR Scientific), and examined by using an Axiovert 200 fluorescence microscope (Carl Zeiss). Fluorescence and differential interference contrast images were captured and processed using AxioVision release 4.6.

Generation of the N-Terminal FLAG-Tagged TEX14, MYC-Tagged CEP55, mCherry-Tagged TX or AAAX3A, and Yellow Fluorescent Protein (YFP)-Tagged Full-Length ALIX Constructs.

The mouse TEX14 open reading frame (ORF) sequence was ligated into the BamHI and SalI sites of the pCMV-tag2 vector (Stratagene), which contains an N-terminal FLAG tag sequence. The mouse CEP55 ORF was cloned from testis cDNA and subcloned into the EcoRI and SalI sites of the pcDNA3 (Invitrogen) vector containing an N-terminal MYC tag sequence. The truncated and mutant TEX14 (TX, AAAX3A) and the full-length ALIX sequences were ligated into the BamHI and NotI or the NotI and XbaI sites, respectively, of the pcDNA3 vectors, while mCherry or YFP sequences were subcloned into the KpnI and BamHI sites. Purified plasmid DNA was obtained by using a QIAprep spin miniprep kit (Qiagen), and all constructs were sequenced for integrity.

Cell Culture and Transfection.

HEK293T or HeLa cells were maintained in Dulbecco's modified Eagle medium (Invitrogen) supplemented with 10% fetal calf serum (SAFC Biosciences), 1% L-glutamine (Invitrogen), and penicillinstreptomycin (Invitrogen) and grown on poly-D-lysine (Sigma)-coated coverslips in culture plates at 37 C° in a humidified 5% CO₂ atmosphere. For immunoprecipitation and immunoblot experiments, cells were seeded at 50 to 80% confluence in 10-cm² dishes (Corning) and transiently transfected using Fugene 6/HD transfection reagent (Roche) according to the manufacturer's instructions.

Communoprecipitation and Western Blot Analysis.

FLAG-TEX14 and/or MYC-CEP55 (3 or 6 μg of DNA for double or single transfection, respectively) were overexpressed in HEK293T cells, as described above. At 48 h after transfections, cells were rinsed with phosphate-buffered saline (PBS) and lysed with M-PER mammalian protein extraction reagent (Pierce), and the lysates were sonicated and divided equally into three tubes. Lysates were incubated with anti-FLAG and anti-MYC antibodies and, as a control, protein G at 4° C. for 1 h to overnight and then incubated with protein G-Sepharose 4B (Sigma) preblocked with 3% BSA-TBS. The immunoprecipitates were washed four times with PBS-1% Triton and once with PBS. The immunoprecipitates and total cell lysates were separated by 3 to 8% Tris-acetate gel (Invitrogen) and transferred onto a nitrocellulose membrane (Protran BA83). Western blot assays were performed using mouse anti-MYC monoclonal antibody (1:5,000; BD Biosciences) and mouse anti-FLAG monoclonal antibody (1:8,000; Sigma) as primary antibodies and horseradish peroxidase-conjugated anti-mouse IgG (1:10,000; Jackson Immunoresearch) as a secondary antibody. Western blot analyses of the enriched intercellular bridge fractions were performed using the affinity-purified goat anti-TEX14 antibody (1:500) and guinea pig anti-CEP55 antibody (1:500) as primary antibodies. Proteins were detected with chemiluminescence by Super-Signal West Pico chemiluminescent substrate (Thermo Scientific) and exposed to BioMax XAR film (Eastman Kodak).

Yeast Two-Hybrid System and Oxygen-Biosensor Assay.

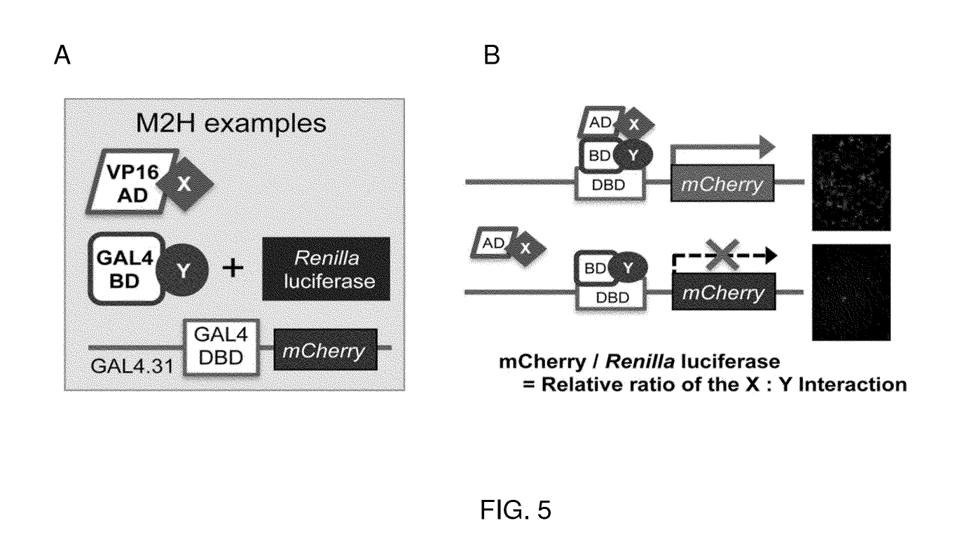

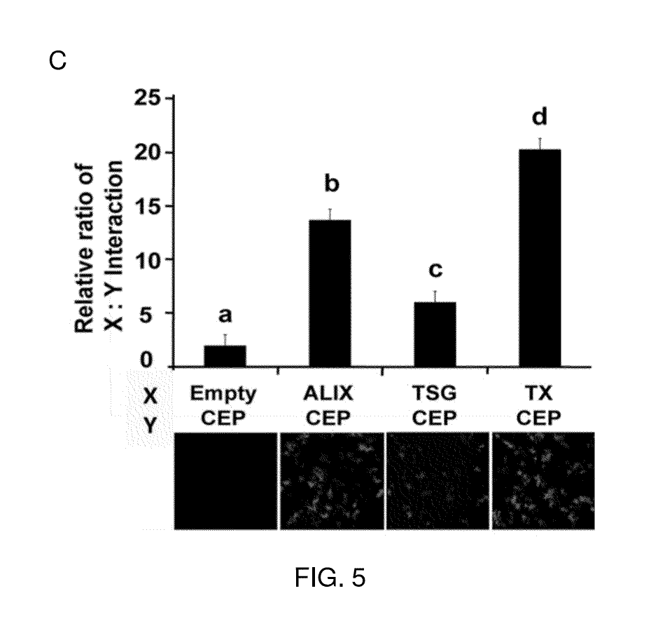

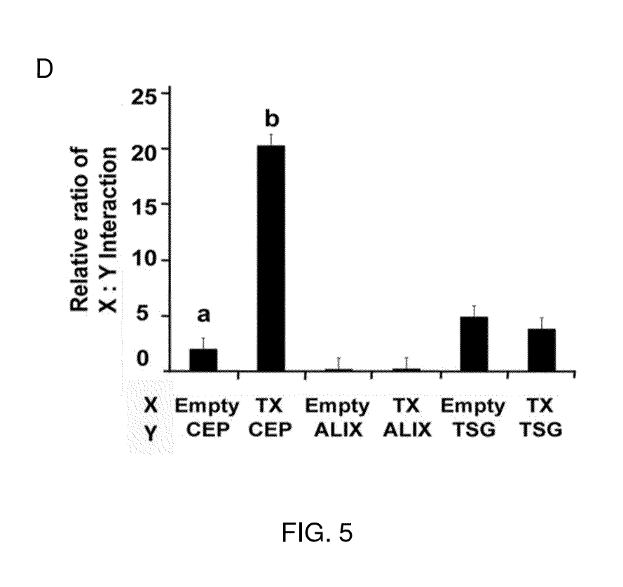

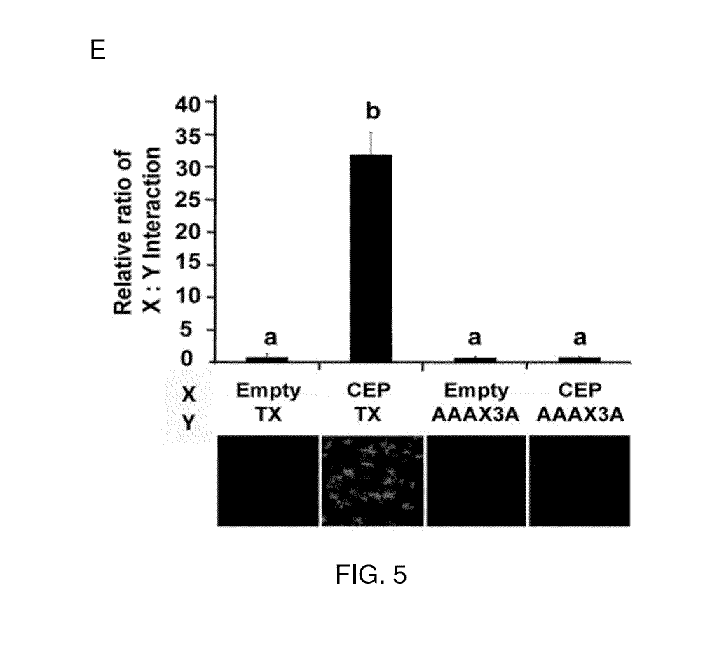

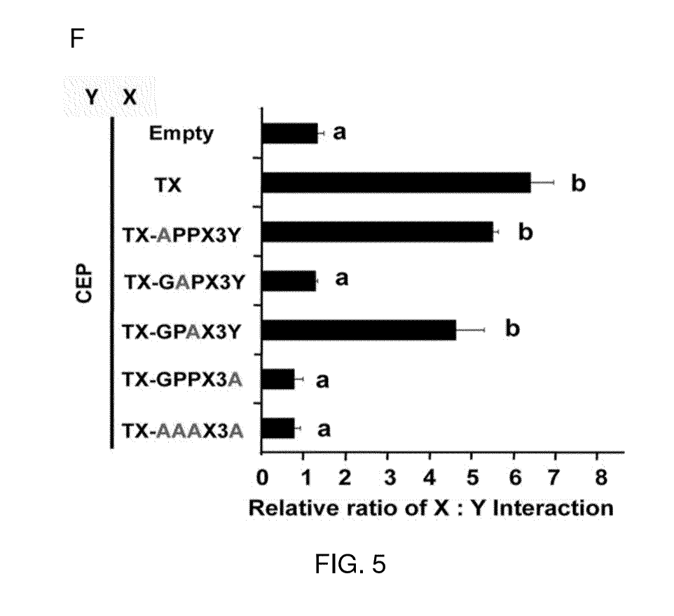

Protein-protein interactions were evaluated by using the Matchmaker Two-Hybrid System 3 (Clontech) as described previously (10). Mouse full-length TEX14 and MLKP1 were previously subcloned into the Matchmaker GAL4 two-hybrid pGBKT7 and pGADT7 vectors (10). The constructs of full-length mouse and human CEP55, human TEX14, truncated mouse CEP55-C1, and truncated mouse TEX14-T1, T2, T1□T2, T3, T4, and T4-C were made by using the Matchmaker GAL4 two-hybrid pGBKT7 bait vector and pGADT7 prey vectors. Some exemplary constructs used are summarized in FIG. 5A. Some Y2H interactions were examined with an oxygen biosensor assay (Clontech PT3584-1) that measured the fluorescence emitted by an oxygen-sensing platform that detects GAL4.

Mammalian Two-Hybrid System.

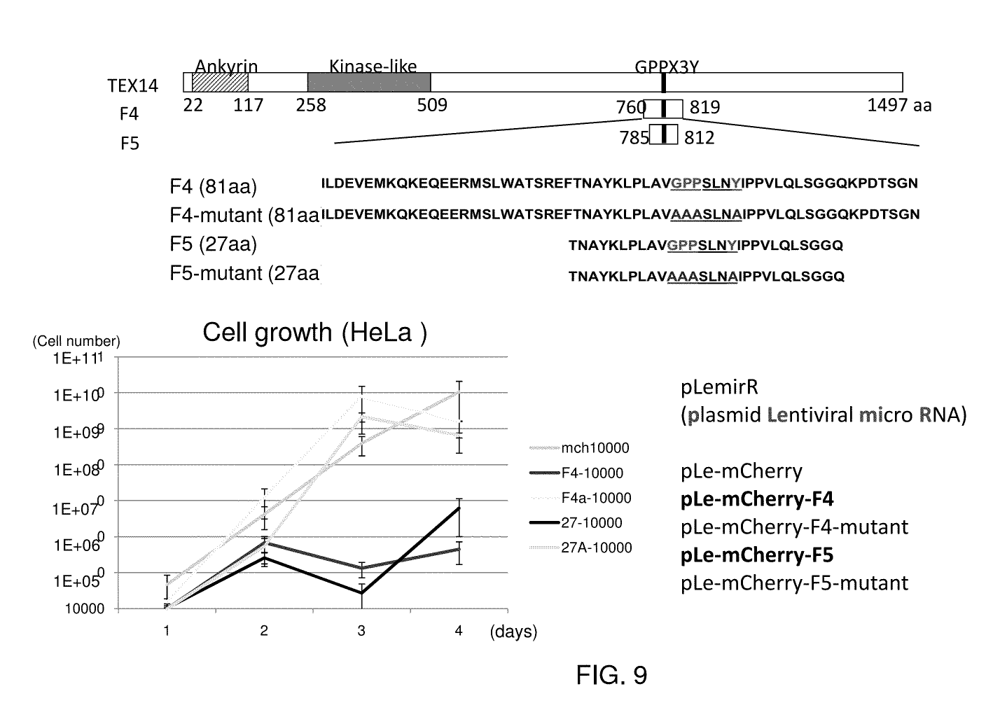

A modified form of the CheckMate/Flexi vector mammalian two-hybrid system (Promega) was used to study the interactions of proteins in mammalian cells. A pGL4Cherry (mCherry/GAL4UAS/Hygro) vector was engineered from the pGL4.31 (luc2P/GAL4UAS/Hygro) vector by replacing the Luc2P firefly luciferase reporter sequence with the mCherry sequence. The pACT vector expresses a protein of interest, “X,” fused to a transcriptional activation domain (VP16-AD) and the pBIND vector expresses the other protein of interest, “Y,” fused to a DNA-binding domain (GAL4-BD) and Renilla luciferase by separate promoters. A pBIND-YFP vector was engineered from the pBIND vector by replacing Renilla luciferase sequence with the YFP sequence for experiments in FIG. 9.

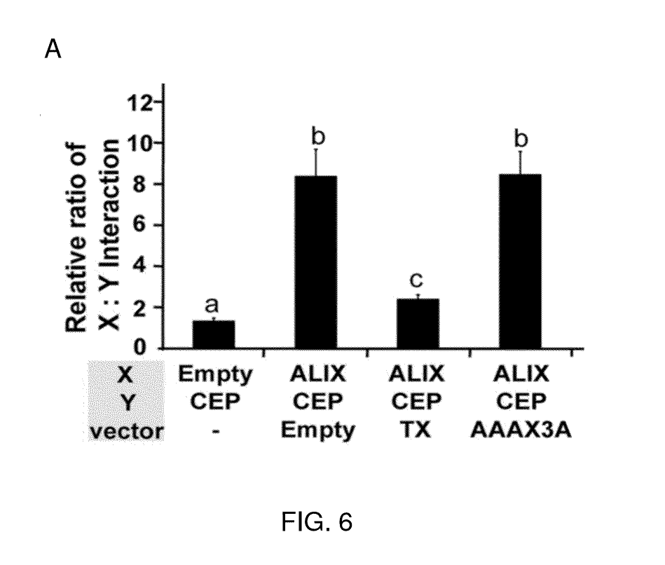

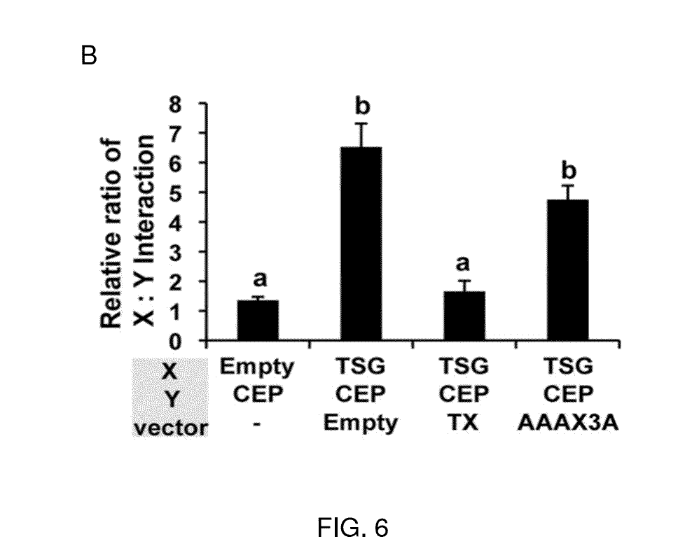

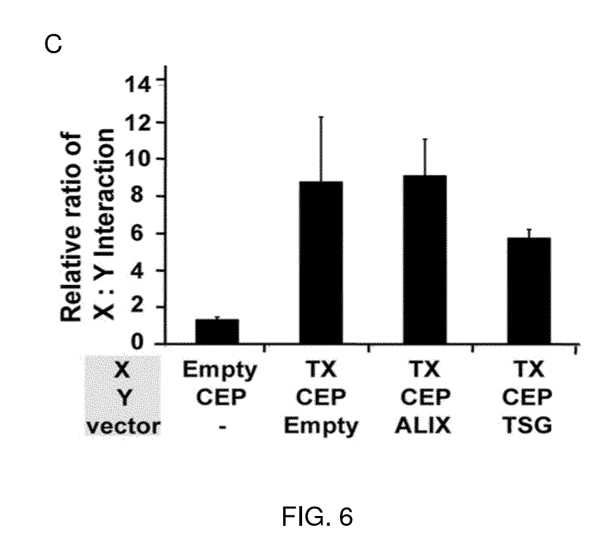

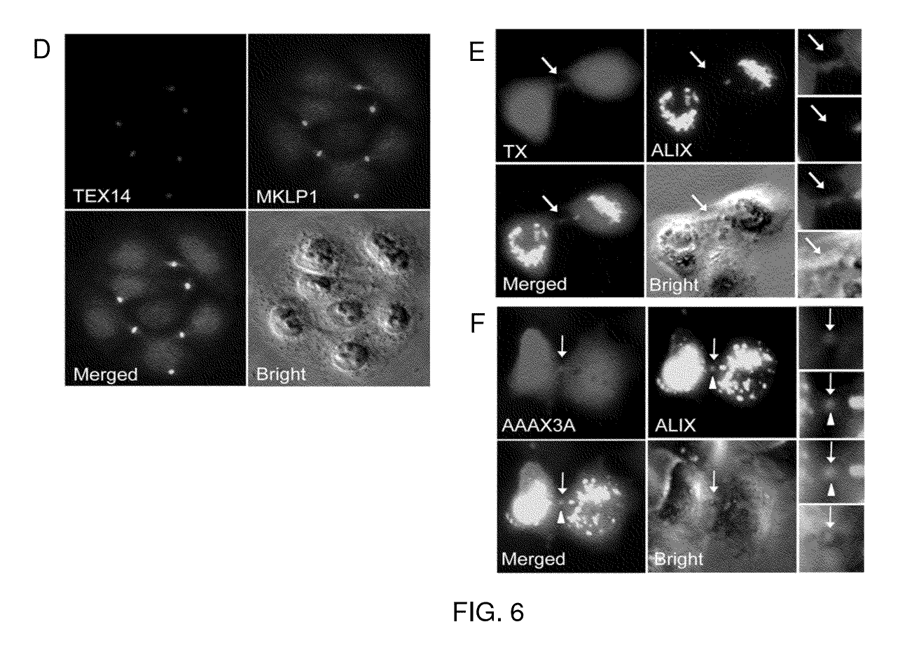

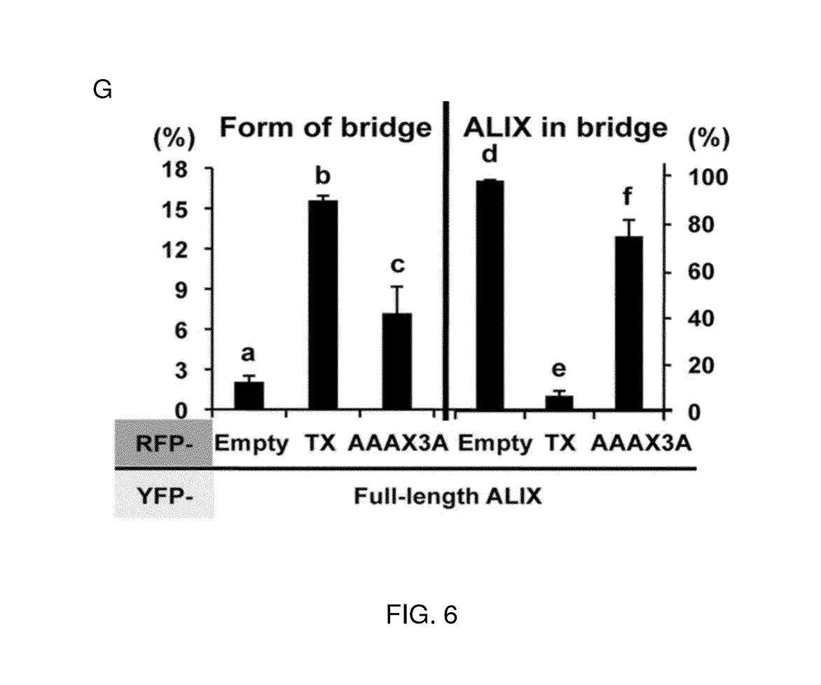

Sequences encoding the truncated TEX14, CEP55, ALIX, or TSG101 fragments were cloned into the multiple cloning regions of the pACT and pBIND vectors. Some exemplary constructs used are summarized in FIGS. 6A and B. pACT (protein X), pBIND (protein Y), and pGL4Cherry (mCherry/GAL4UAS/Hygro) were transiently cotransfected into HEK293T cells using Fugene 6/HD transfection reagent, and the cells were examined 44 h later for red fluorescence by using an Axiovert 40 CFL microscope (Carl Zeiss). AxioVision release 4.6 was used for analysis of the image. The pACT-MyoD and pBIND-Id vectors served as positive controls, whereas the empty pACT and pBIND vectors served as negative controls for protein interaction. The cells were lysed by using Renilla luciferase assay reagent (Promega), and red fluorescence and Renilla luciferase were measured by using a Polar Star Omega microplate reader (BMG LabTech). The excitation and emission wavelengths were 580 and 610 nm, respectively, for red fluorescence (mCherry), 480 and 520 nm for yellow fluorescence (YFP), and luminescence for Renilla luciferase. Protein interactions were quantified by using the ratio of red fluorescence divided by Renilla luciferase or yellow fluorescence.

Competition Assays Using the Mammalian Two-Hybrid Assay System.

The truncated TEX14 (TX), TEX14 AAAX3A mutant (AAAX3A; SEQ ID NO:3), ALIX (ALIX), and TSG101 (TSG) pcDNA3 overexpression vectors were produced and expressed in HEK293T cells with mammalian two-hybrid vectors, pGL4Cherry (mCherry/GAL4UAS/Hygro), the VP16-AD-X and the GAL4-BD-Y vectors. Empty pcDNA3 vector was transfected as a control. Some exemplary constructs used are summarized in FIGS. 6A and B, procedure B.

Alignment of the Motif and Flanking Sequences from TEX14 Orthologs.

By database mining using UCSC Genome Browser (see the world wide web), NCBI (see the world wide web), and Ensembl (see the world wide web), the protein sequences for TEX14 orthologs were obtained directly or deduced from ORFs of predicted cDNAs assembled from expressed sequence tags. Full-length Xenopus laevis and chicken (Gallus gallus) Tex14 cDNAs were cloned using 5′ and 3′ RACE (rapid amplification of cDNA ends), using the SMART RACE cDNA amplification kit (BD Biosciences). The following gene-specific primers were used: XlTex14-3RACE, 5′-CGTTGGTCAGCCCAGAAGTCATCA (SEQ ID NO:4); XlTex14-5RACE, 5′-GGGC CCAGCAGTATGGTTTCCTACA (SEQ ID NO:5); XlTex14-INT-F, 5′-CTTTTCTGGGAACCA GTGGAGT (SEQ ID NO:6); XlTex14-INT-R, 5′-TTATGGCCAGAAATCACTGCAT (SEQ ID NO:7); gTex14-5RACE, 5′-ACAGCGATGGTTAGGGTCTGAG (SEQ ID NO:8); gTex14-3RACE, 5′-GGACCT AGACCACGAGCATTTA (SEQ ID NO:9); gTex14-Ext-F, 5′-CGCTGTTAGGCCTTGGTAAGAT (SEQ ID NO:10); and gTex14-Ext-R, 5′-TCTTCTTTGCTTCCACTTGCTG (SEQ ID NO:11). The ORFs of deduced and cloned cDNAs were determined by using the EditSeq program of DNAStar (Madison, Wis.).

Statistical Analysis.

Data were analyzed by analysis of variance according to the two-tailed Student t test. Differences between the mean values were considered to be statistically significant at P<0.05.

### Example 2

### TEX14 Interacts with CEP55 to Block Cell Abscission

CEP55 is a Component of Stable Intercellular Bridge in Testis and Ovary.

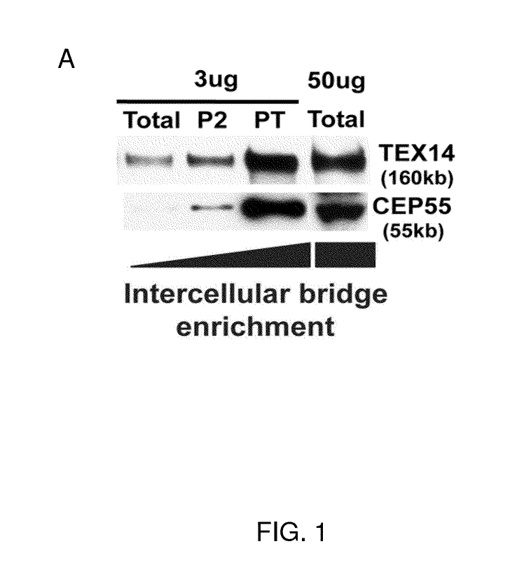

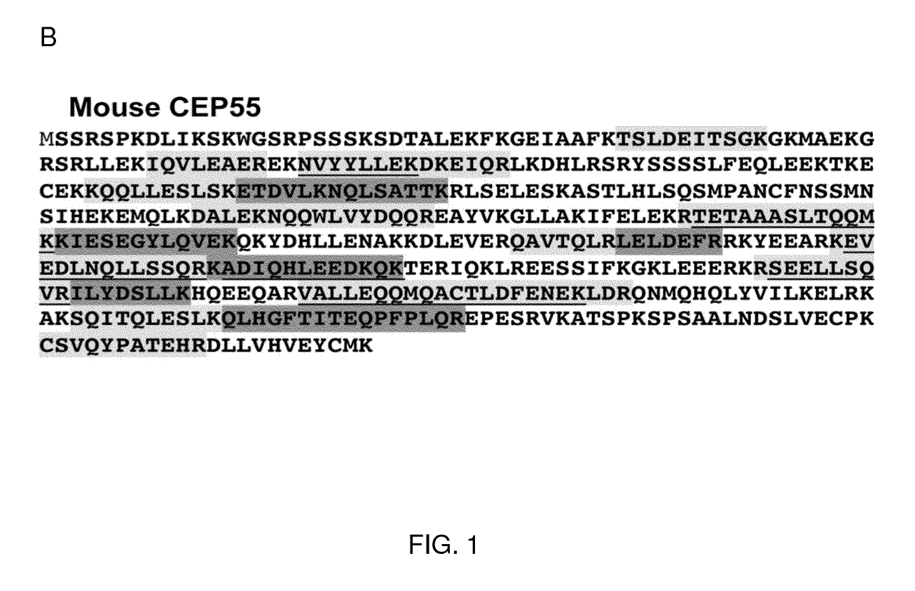

To identify components of the mammalian intercellular bridge and determine how the bridge forms, the inventors developed a biochemical method to enrich intercellular bridges from mouse testes using TEX14 as a major marker protein (FIG. 1A) (10). This material was fractionated by size and subjected to liquid chromatography-tandem mass spectrometry proteomic analysis. In addition to TEX14, the inventors isolated 19 proteins that have roles in cytokinesis, including the centralspindlin complex, MKLP1, and MgcRacGap (10). CEP55 mRNA was previously shown to be highly expressed in the testis (6, 15), and CEP55 protein was also identified with 24 peptides in our bridge preparation (FIG. 1B). To confirm that CEP55 was a component of the intercellular bridge and not a contaminant, the inventors performed Western blot analyses of the enriched intercellular bridges and immunofluorescence of the purified intercellular bridge preparations. CEP55 and TEX14 are coenriched by this biochemical method and colocalize in the purified intercellular bridges (at least FIG. 1A). To analyze the localization of CEP55 protein in immature and adult testes, immunofluorescence analysis was performed with antibodies generated to mouse TEX14 and mouse CEP55. Consistent with the purified bridge results, CEP55 and TEX14 perfectly colocalize as ring-shaped intercellular bridge structures in germ cells throughout the seminiferous tubules and at all stages of spermatogenesis. The bridge diameter expands from the juvenile to the adult stage, indicating that additional CEP55 and TEX14 are added to the intercellular bridge during spermatogenesis. In the female, CEP55 is expressed and colocalizes with TEX14 in embryonic day 18.5 mouse ovary.

TEX14 Interacts with CEP55.

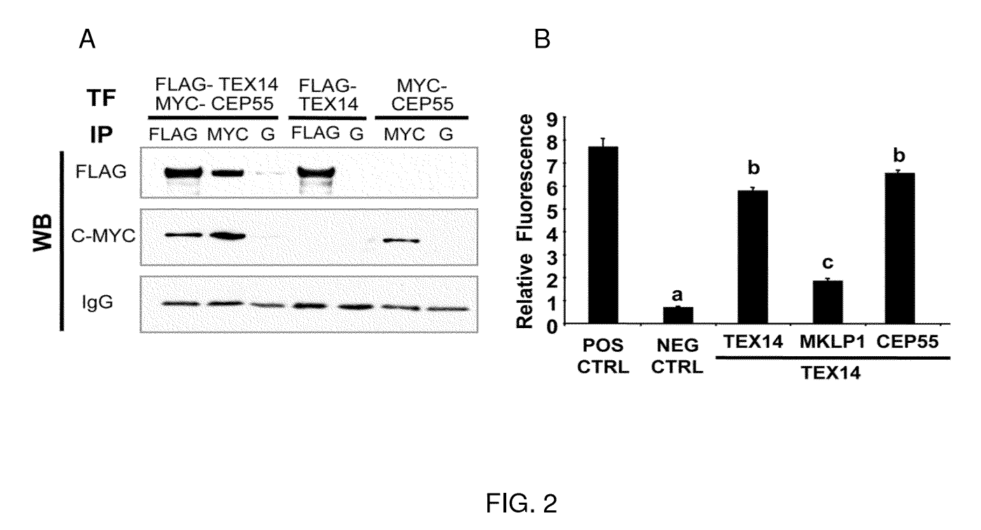

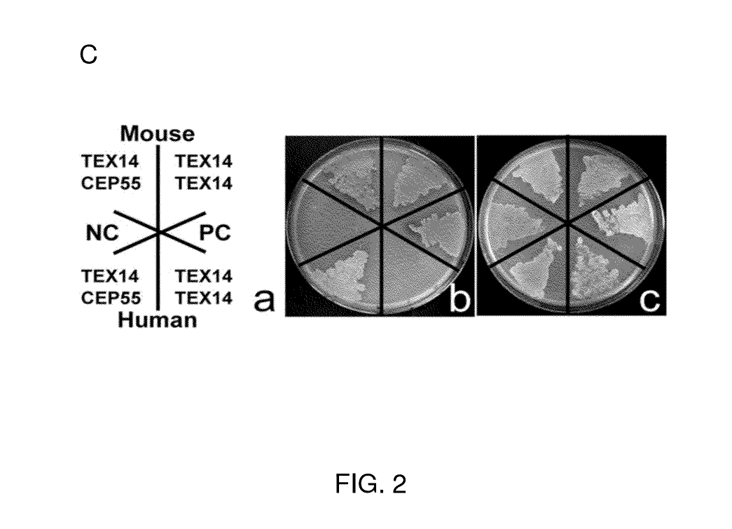

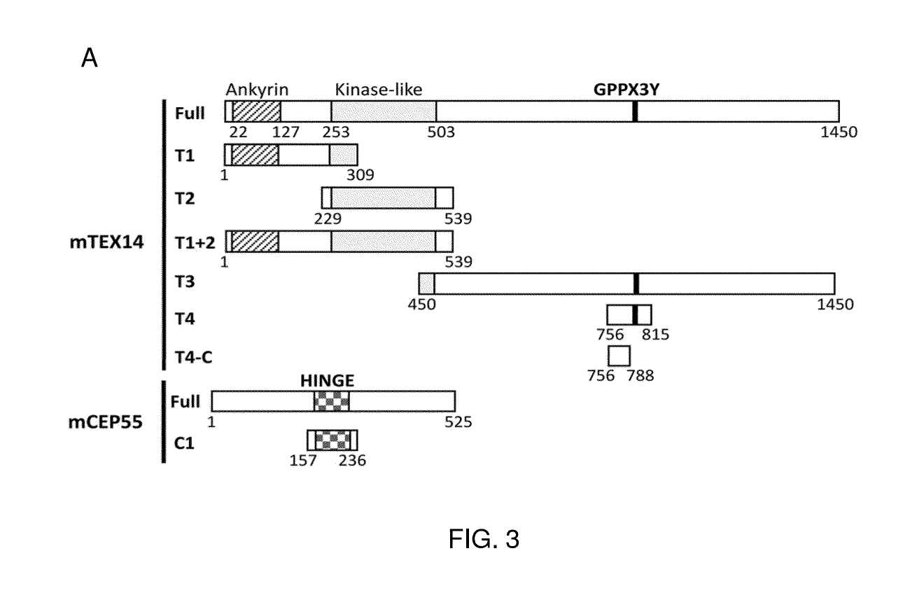

Since both CEP55 and TEX14 interact with MKLP1 (10, 22), it was determined whether CEP55 and TEX14 are components of the intercellular bridge as direct or indirect interactors. The inventors first overexpressed full-length FLAG-TEX14 and MYC-CEP55 constructs in HEK293T cells (FIG. 2A). Immunoprecipitation with anti-FLAG antibody, followed by Western blot analysis with anti-FLAG or anti-MYC antibodies, reveals that the inventors not only immunoprecipitate FLAGTEX14 but also MYC-CEP55. Likewise, when the inventors immunoprecipitate with anti-MYC antibody, they immunoprecipitate both MYC-CEP55 and FLAG-TEX14. When the inventors express only FLAG-TEX14 and immunoprecipitate with anti-FLAG antibody, the anti-MYC antibody does not cross-react and conversely when the inventors only express MYC-CEP55 and immunoprecipitate with anti-MYC antibody, the anti-FLAG antibody does not cross-react. Thus, by coimmunoprecipitation, the inventors are able to show that TEX14 and CEP55 form a complex. To determine the intensity of the interactions of TEX14 and CEP55, the inventors first performed yeast two-hybrid assays with full-length mouse or human TEX14 and CEP55. Mouse TEX14 interacts strongly with itself and CEP55 and more weakly with MKLP1 (FIGS. 2B and C). Human TEX14 and CEP55 also interact, but, oddly, human TEX14 does not in-teract with itself in this assay (FIG. 2C). Thus, mammalian orthologs of TEX14 and CEP55 interact strongly.

TEX14 has a GPPX3Y Motif.

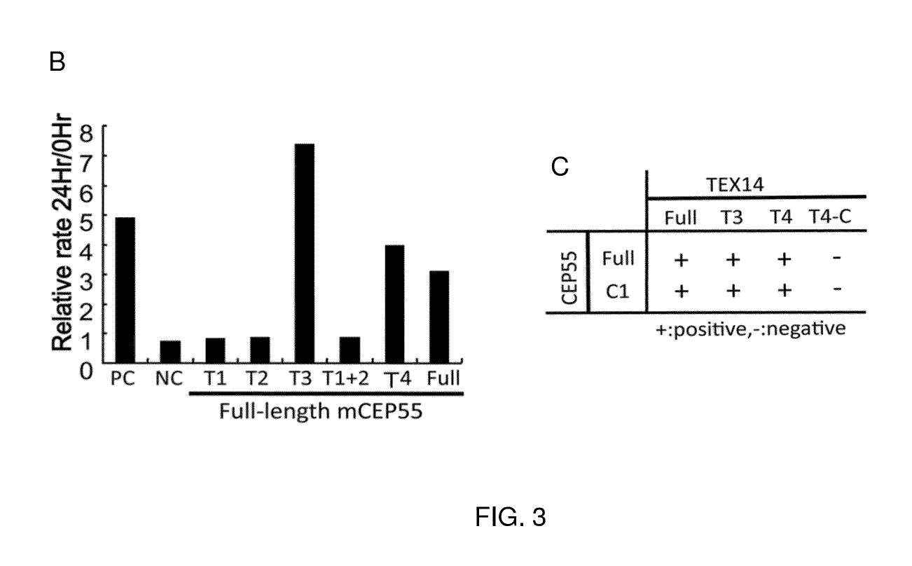

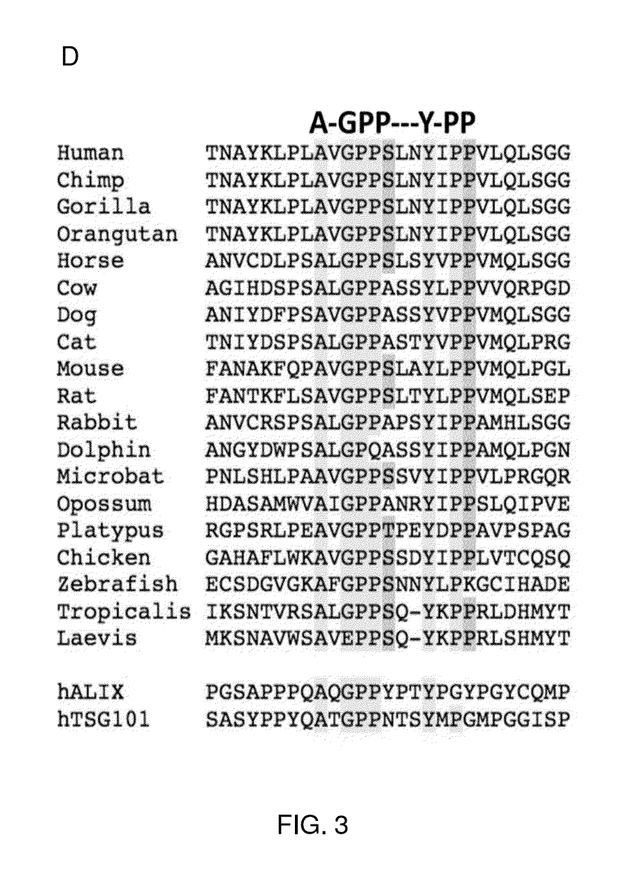

To define the core regions of TEX14 and CEP55 that interact, the inventors constructed yeast two hybrid vectors to express full-length and truncated mouse TEX14 and CEP55 proteins (FIG. 3A). TEX14 has three ankyrin repeats, a kinaselike domain, and a C-terminal leucine zipper dimerization motif (21) (FIG. 3A). Whereas full-length TEX14 interacts with full-length CEP55, N-terminal truncations of TEX14 that contained the ankyrin repeats, the kinaselike domains, or both failed to interact with full-length CEP55 (FIG. 3B). However, the long TEX14 C-terminal domain that contains several coiled-coil domains interacts with full-length CEP55 (FIG. 3B).

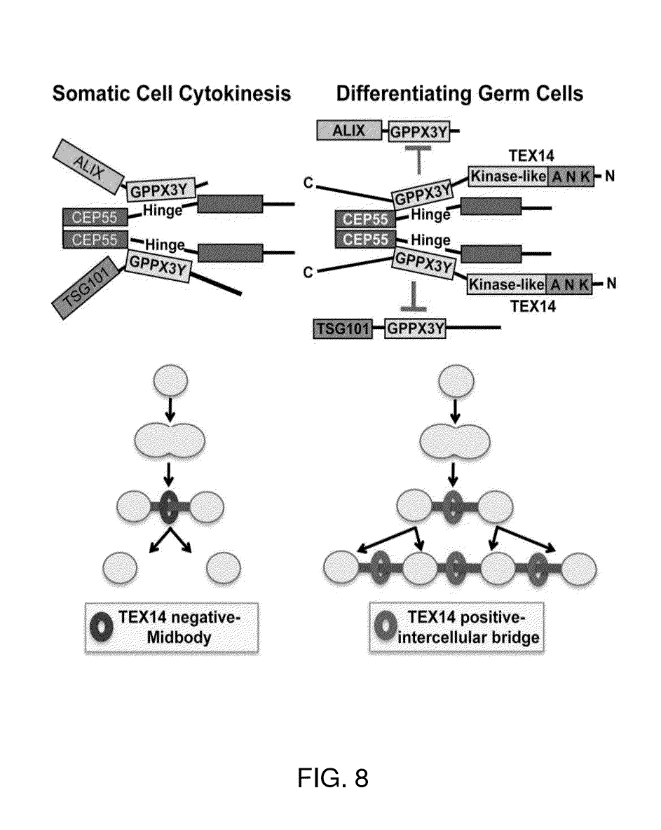

In somatic cells, CEP55 dimerizes through coiled-coil domains at the N terminus (17, 22) (see FIG. 8, left). During cytokinesis, GPPX3Y-containing coiled-coil domains of ALIX and TSG101 bind to the “hinge” region of CEP55 (14, 17) (FIGS. 6A and B and see FIG. 11, left). These interactions are essential for somatic cell abscission (2, 3, 17). Because the C-terminal region of TEX14 contains coiled-coil domains and interacts with full-length CEP55 (FIGS. 3B and C), the inventors searched for similar GPPX3Y domains in TEX14 that could be mimicking the ALIX and TSG101 interactions with CEP55. In addition to previously published mouse and human TEX14 orthologs (20, 21), the inventors cloned the Xenopus laevis and Gallus gallus orthologs and uncovered an additional 15 full-length or partial TEX14 sequences in the public database. Although there is significant divergence of these orthologs in their C-terminal regions compared to the ankyrin repeats and kinase-like domains in their N termini, 17 of the 19 proteins share a similar GPPX3Y motif in their C termini, with the exceptions being the Xenopus laevis and dolphin orthologs (FIG. 3D). Analysis of the TEX14 sequences in and around the GPPX3Y motif demonstrated a conserved alanine upstream of the motif that is also shared with the human ALIX and TSG101 sequences, a conserved serine/threonine/alanine within the motif, and two conserved prolines downstream, one of which is also present in TSG101.

The Region Including the GPPX3Y Motif of Mouse and Human TEX14 Interacts with the Hinge of CEP55.

To determine the relevance of this GPPX3Y motif as it relates to the interaction of TEX14 with CEP55, we performed yeast two-hybrid assays with mouse TEX14 amino acids 756 to 815 that contain the GPPX3Y motif (FIG. 3A). These 60 amino acids interacted with full-length CEP55 (FIGS. 3B and C). Conversely, an 80-amino-acid fragment of CEP55 (C1) that contains the hinge region interacted with full-length TEX14, the C terminus (T3), and GPPX3Y-containing fragment (T4) but not a shorter sequence that lacked the GPPX3Y motif (T4-C) (FIG. 3A to C). Thus, similar GPPX3Y-containing regions of TEX14, ALIX, and TSG101 interact with the hinge region of CEP55.

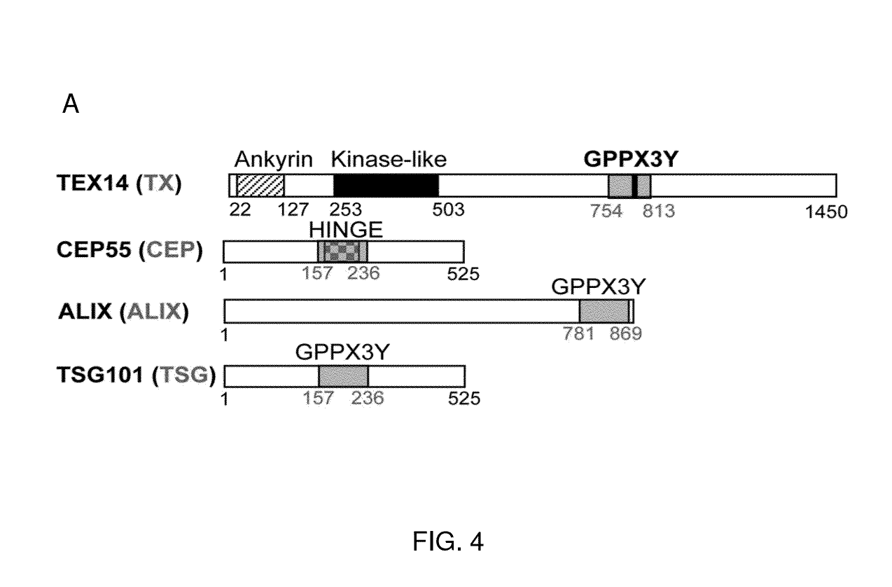

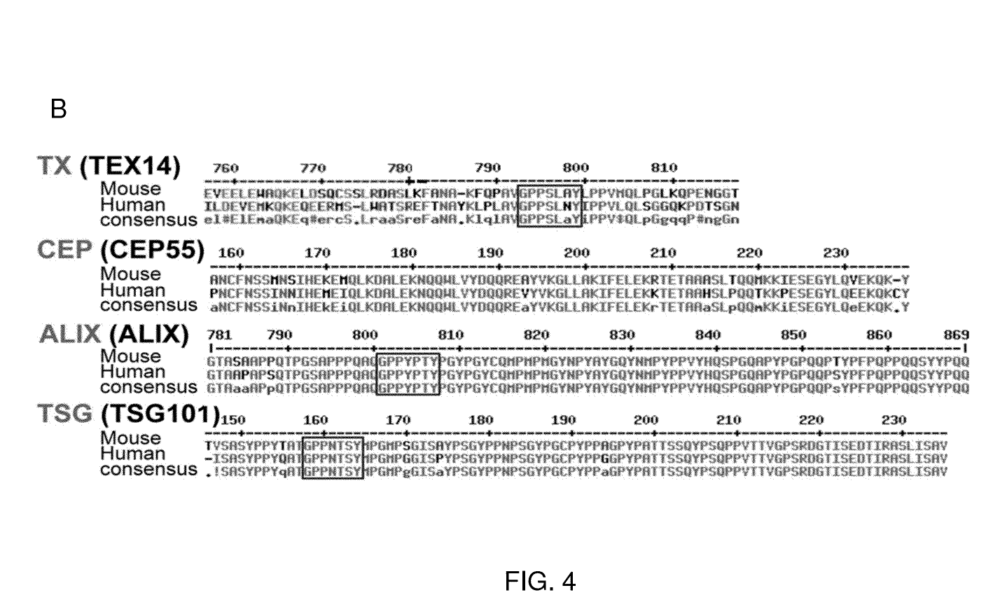

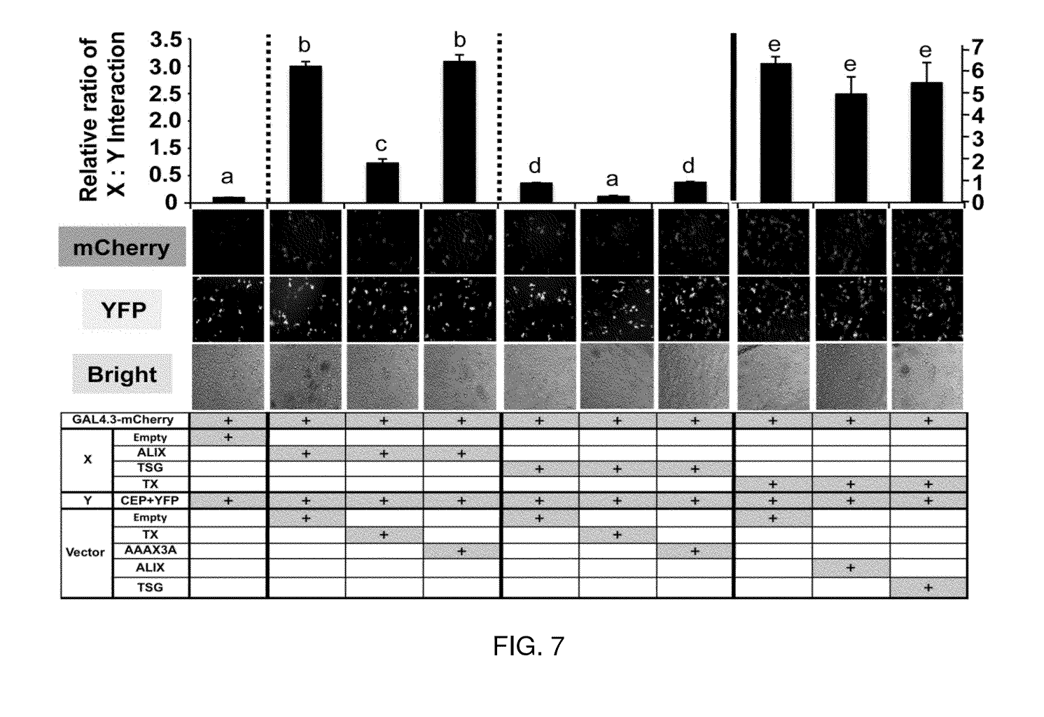

Based on these expression, colocalization, and yeast twohybrid interaction data, we hypothesized that the conserved GPPX3Y motif of TEX14 (FIG. 3D) interacts strongly with the hinge region of CEP55 and blocks the interaction of CEP55 with TSG101 and ALIX (see FIG. 11, right). To further characterize this embodiment, the inventors developed several modified mammalian two-hybrid assays to study these protein-protein interactions in a more natural mammalian cell setting. When the expressed proteins “X” and “Y” interact, the mCherry protein can be detected as red fluorescence (FIGS. 5A and B). The GAL4-BD (“Y”) vector expresses Renilla reniformis luciferase (FIG. 5C to F and FIG. 6A to C) or yellow fluorescence (YFP) (FIG. 7) under the control of the simian virus 40 (SV40) promoter, normalizing for differences in transfection efficiency. Key regions of mouse and human TEX14 (TX), CEP55 (CEP), ALIX (ALIX), and TSG101 (TSG) (FIGS. 4A and B) were cloned in frame into these vectors and cotransfected into HEK293T cells. Similar to previous reports (14, 17), the inventors confirmed that the regions including the GPPX3Y motifs of human TSG101 and ALIX interact with the hinge region of human CEP55 (FIG. 5C). Similar to the yeast two-hybrid data, the GPPX3Y region of human TEX14 (TX) (the 60 amino acids from 754 to 813, FIGS. 4A and B) strongly binds to the hinge region of human CEP55 in the mammalian two-hybrid assay (FIG. 5C). In all three cases, intracellular red fluorescence was also detected (FIG. 5C, bottom). In contrast, TEX14 does not interact with ALIX and, in the case of TSG101, TEX14 binding is not significantly different than the TSG101 interaction with empty pACT vector as a control (FIG. 5D).

The GPPX3Y Domain of TEX14 and the First P and Y of GPPX3Y are Essential for Binding with the Hinge of CEP55.

To define how the TEX14 GPPX3Y motif functions during the binding of TEX14 with CEP55, we generated the alanine mutant, AAAX3A. The TEX14 mutant AAAX3A is unable to bind to CEP55 (FIGS. 5E and F). To precisely define the amino acids in the TEX14 GPPX3Y motif that are most required for the interaction with CEP55, we mutated individual glycine (G), proline (P), and tyrosine (Y) residues to alanines. Specific mutations in the first proline (i.e., GAPX3Y) and the tyrosine (i.e., GPPX3A) of the motif are sufficient to abolish TEX14 interaction with CEP55 (FIG. 5F). Consistent with the lack of conservation of the glycine in the Xenopus laevis ortholog of TEX14 (i.e., a G-to-E substitution) (FIG. 3D), mutation of this glycine to alanine did not alter the interaction of human TEX14 with CEP55. It is unclear whether the presence of the glutamine (Q) in the dolphin ortholog (i.e., GPQX3Y) functions like a proline in interactions with CEP55 or is a result of a genomic sequencing error.

The GPPX3Y Motif of TEX14 Inhibits the Interaction of Truncated CEP55 with ALIX or TSG101.

Based on an exemplary model (see FIG. 8, right), in certain embodiments of the invention, TEX14 competes more efficiently for the CEP55 hinge region than either ALIX or TSG101. To further characterize this embodiment, the inventors first analyzed the ALIXCEP55 and TSG101-CEP55 interactions when TEX14 was overexpressed. Whereas ALIX and TSG101 demonstrate a strong interaction with CEP55 in the mammalian two-hybrid assay when an empty pcDNA vector is cotransfected, overexpression of TEX14 (TX) in this setting suppressed both the ALIX-CEP and the TSG-CEP interactions (FIGS. 6A and B, FIG. 7 [left and center]). However, overexpression of the TEX14 mutant AAAX3A does not block either the ALIXCEP or the TSG-CEP interaction (FIGS. 6A and B, FIG. 7 [left and center]), confirming that the GPPX3Y motif is important for the interaction with CEP55 and also essential for the antagonism of ALIX and TSG101 interactions with CEP55. In contrast, overexpression of ALIX and TSG fails to disrupt the TX-CEP interaction (FIG. 6C, FIG. 7 [right]). In addition, the TX-CEP interaction was typically stronger than the ALIX-CEP or TSG-CEP interactions (FIG. 7).

Full-Length TEX14 Localizes to the Midbody and Blocks Cell Abscission.

When full-length TEX14 is overexpressed in HeLa cells, many interconnected cells are visualized with anti-TEX14 and anti-MKLP1 antibodies by immunofluorescence (FIG. 6D). These data indicate that overexpressed TEX14 binds with CEP55 and blocks endogenous HeLa cell-produced ALIX and TSG101 from interacting with CEP55, resulting in inhibition of the completion in cytokinesis and stabilization of a transient intercellular bridge.

The GPPX3Y Motif of TEX14 Inhibits Entry of Full-Length ALIX into the Midbody.

In the testes of mice lacking TEX14, male germ cells continue to divide and enter meiosis, independent of the intercellular bridges. However, these germ cells quickly die at the pachytene spermatocyte stage (11). To characterize how the expression of TEX14 prevents these germ cells from dividing and stabilizes the intercellular connection, the inventors transfected our various constructs into mammalian cells. The inventors focused on ALIX because of its strong interactions with CEP55 in our assays (FIG. 5C, FIG. 6A, and FIG. 7, left) and on the truncated TEX14 to confirm that the GPPX3Y motif of TEX14 inhibits the interaction of ALIX:CEP55. They generated pcDNA-mCherry-truncated TEX14 (wild-type TX or mutant AAAX3A) and pcDNA-YFP full-length ALIX overexpression vectors and cotransfected them into HeLa cells. The truncated TEX14 and AAAX3A mutant are overexpressed throughout the cells, including the midbody, but do not specifically localize to the midbody like full-length TEX14 (FIG. 6D) because the truncated TEX14 and AAAX3A mutant are only 60 amino acids and do not have the other critical domains except the GPPX3Y motif (FIG. 4). The truncated GPPX3Y TEX14 (TX) inhibits full-length ALIX localization to the midbody (FIGS. 8E and G, right), and several interconnecting cells were visualized in the TX transfected cells similar to the full-length TEX14-transfected cells in FIG. 6D (FIG. 6G, left). In contrast, fulllength ALIX continues to localize to the midbody when the truncated TEX14 mutant (AAAX3A) construct is cotransfected (FIGS. 6F and G, right), similar to the control experiment when the pcDNA-mCherry empty vector is transfected along with ALIX. Thus, TEX14 blocks the ability of ALIX to interact with endogenous CEP55, preventing abscission, and instead TEX14-CEP55 complexes contribute to the formation of stable intercellular bridges (FIGS. 6D and E).

The inventors defined the GPPX3Y truncated TEX14 (F4, 81aa) is useful to form the intercellular bridges and that GPPX3Y motif is at least an example of a core region for the function. In specific embodiments, the peptide is useful to inhibit the growth of cancer cells, and cell growth is characterized with a variety of TEX14 peptide. The inventors made an exemplary smaller peptide (F5, 27aa; FIG. 9). F4 or F5 mutants were prepared as negative controls, respectively, wherein they have Alanine on behalf of G, P, P, and Y. These peptides (F4, F4-mutant, F5, and F5-mutant) are fusion proteins with mCherry and were over expressed by lentivirus vector. At first, the inventors examined HeLa cells as representative cancer cells. The control cells that were expressed grew very well (mcherry; dark blue). F4-mutant and F5-mutant expressed cells also grew very well, like the control experiments (F4-mutant; green, F5-mutant; light blue). F4 and F5-expressed cells did not grow (F4; red, F5; purple). This, in some embodiments the TEX14 peptide is useful having a length of about 30 amino acids.

In summary, the studies show that the GPPX3Y containing region of TEX14 binds to the hinge region of CEP55 and inhibits the interactions of ALIX and TSG101 with CEP55 (FIG. 5C to F, FIGS. 6A and B, and FIG. 7). The studies involving mutations in the GPPX3Y motif and disruption of the interactions between CEP55 and ALIX or TSG101 argue strongly for this to be a direct interaction. In the exemplary model (see FIG. 8), the steps involved in abscission normally are MKLP1→CEP55→ALIX/TSG101→˜→abscission. However, in differentiating male (and presumably female) germ cells, the steps involved appear to be MKLP1→TEX14→CEP55→intercellular bridge. When GPPX3Y containing TEX14 fragments are present (FIGS. 5E and F), ALIX does not enter the midbody, indicating that TEX14 prevents the completion of cytokinesis, including the localization of ALIX, by altering the fate of CEP55 from a midbody organizer protein that recruits proteins for abscission to the midbody to a major component in the stable intercellular bridge, in certain embodiments. One additional mechanism by which TEX14 could more effectively compete for CEP55 is through its interaction with MKLP1. TEX14 appears early in the process of cytokinesis by binding MKLP1 and “filling” the intercellular bridge. At a later point, MKLP1, MgcRacGap, and TEX14 are overlapping in a single structure. In certain embodiments, it is at this point that CEP55 approaches the midbody and is bound tightly and simultaneously by both MKLP1 and TEX14, never allowing the recruitment of ALIX or TSG101 for abscission.

Thus, in male (and presumably female) germ cells, through a combination of a high local concentration of TEX14 in the transient bridge and a stronger TEX14-CEP55 interaction relative to the CEP55-ALIX or CEP55-TSG101 interactions (FIG. 7), ALIX and TSG101 are not recruited to the midbody and are unable to bind with CEP55 to complete cytokinesis (see FIG. 8, right). In the testes of TEX14 knockout mice, CEP55 localizes to the midbody and, in the absence of local TEX14 to interact, ALIX and TSG101 are permitted access to CEP55 to complete cytokinesis, disrupting the transient intercellular bridge and resulting in an eventual failure of spermatogenesis (FIG. 8, left) (11).

CEP55 mRNA and protein are increased in a number of tumor cell lines and carcinoma tissues (13, 18), and CEP55 is part of a 70-gene signature of chromosomal instability that is predictive of decreased survival in several cancer types (4). These findings indicate that interfering with CEP55 function in tumor cells is a useful therapeutic strategy for targeting multiple different types of cancer. In certain embodiments of the invention, peptides containing the GPPX3Y motif of TEX14 function as antineoplastic agents, capable of suppressing cancer cell proliferation by binding with CEP55 and generating stable intercellular bridges, for example.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
1. Braun, R. E., R. R. Behringer, J. J. Peschon, R. L. Brinster, and R. D. Palmiter. 1989. Genetically haploid spermatids are phenotypically diploid. Nature 337:373-376.
2. Carlton, J. G., M. Agromayor, and J. Martin-Serrano. 2008. Differential requirements for Alix and ESCRT-III in cytokinesis and HIV-1 release. Proc. Natl. Acad. Sci. U.S.A. 105:10541-10546.
3. Carlton, J. G., and J. Martin-Serrano. 2007. Parallels between cytokinesis and retroviral budding: a role for the ESCRT machinery. Science 316:1908-1912.
4. Carter, S. L., A. C. Eklund, I. S. Kohane, L. N. Harris, and Z. Szallasi. 2006. A signature of chromosomal instability inferred from gene expression profiles predicts clinical outcome in multiple human cancers. Nat. Genet. 38:1043-1048.
5. Dym, M., and D. W. Fawcett. 1971. Further observations on the numbers of spermatogonia, spermatocytes, and spermatids connected by intercellular bridges in the mammalian testis. Biol. Reprod. 4:195-215.
6. Fabbro, M., B. B. Zhou, M. Takahashi, B. Sarcevic, P. Lal, M. E. Graham, B. G. Gabrielli, P. J. Robinson, E. A. Nigg, Y. Ono, and K. K. Khanna. 2005. Cdk1/Erk2- and Plk1-dependent phosphorylation of a centrosome protein, Cep55, is required for its recruitment to midbody and cytokinesis. Dev. Cell 9:477-488.
7. Fawcett, D. W., S. Ito, and D. Slautterback. 1959. The occurrence of intercellular bridges in groups of cells exhibiting synchronous differentiation. J. Biophys. Biochem. Cytol. 5:453-460.
8. Glotzer, M. 2005. The molecular requirements for cytokinesis. Science 307: 1735-1739.
9. Greenbaum, M. P., N. Iwamori, J. E. Agno, and M. M. Matzuk. 2009. Mouse TEX14 is required for embryonic germ cell intercellular bridges but not female fertility. Biol. Reprod. 80:449-457.
10. Greenbaum, M. P., L. Ma, and M. M. Matzuk. 2007. Conversion of midbodies into germ cell intercellular bridges. Dev. Biol. 305:389-396.
11. Greenbaum, M. P., W. Yan, M. H. Wu, Y. N. Lin, J. E. Agno, M. Sharma, R. E. Braun, A. Rajkovic, and M. M. Matzuk. 2006. TEX14 is essential for intercellular bridges and fertility in male mice. Proc. Natl. Acad. Sci. U.S.A. 103:4982-4987.
12. Huckins, C. 1978. Spermatogonial intercellular bridges in whole-mounted seminiferous tubules from normal and irradiated rodent testes. Am. J. Anat. 153:97-121.
13. Inoda, S., Y. Hirohashi, T. Torigoe, M. Nakatsugawa, K. Kiriyama, E. Nakazawa, K. Harada, H. Takasu, Y. Tamura, K. Kamiguchi, H. Asanuma, T. Tsuruma, T. Terui, K. Ishitani, T. Ohmura, Q. Wang, M. I. Greene, T. Hasegawa, K. Hirata, and N. Sato. 2009. Cep55/c10orf3, a tumor antigen derived from a centrosome residing protein in breast carcinoma. J. Immunother. 32:474-485.
14. Lee, H. H., N. Elia, R. Ghirlando, J. Lippincott-Schwartz, and J. H. Hurley. 2008. Midbody targeting of the ESCRT machinery by a noncanonical coiled coil in CEP55. Science 322:576-580.
15. Martinez-Garay, I., A. Rustom, H. H. Gerdes, and K. Kutsche. 2006. The novel centrosomal associated protein CEP55 is present in the spindle midzone and the midbody. Genomics 87:243-253.
16. Mishima, M., S. Kaitna, and M. Glotzer. 2002. Central spindle assembly and cytokinesis require a kinesin-like protein/RhoGAP complex with microtubule bundling activity. Dev. Cell 2:41-54.
17. Morita, E., V. Sandrin, H. Y. Chung, S. G. Morham, S. P. Gygi, C. K. Rodesch, and W. I. Sundquist. 2007. Human ESCRT and ALIX proteins interact with proteins of the midbody and function in cytokinesis. EMBO J. 26:4215-4227.
18. Sakai, M., T. Shimokawa, T. Kobayashi, S. Matsushima, Y. Yamada, Y. Nakamura, and Y. Furukawa. 2006. Elevated expression of C10orf3 (chromosome 10 open reading frame 3) is involved in the growth of human colon tumor. Oncogene 25:480-486. 19. Ventela, S., J. Toppari, and M. Parvinen. 2003. Intercellular organelle traffic through cytoplasmic bridges in early spermatids of the rat: mechanisms of haploid gene product sharing. Mol. Biol. Cell 14:2768-2780.
20. Wang, P. J., J. R. McCarrey, F. Yang, and D. C. Page. 2001. An abundance of X-linked genes expressed in spermatogonia. Nat. Genet. 27:422-426.
21. Wu, M. H., A. Rajkovic, K. H. Burns, W. Yan, Y. N. Lin, and M. M. Matzuk. 2003. Sequence and expression of testis-expressed gene 14 (Tex14): a gene encoding a protein kinase preferentially expressed during spermatogenesis. Gene Expr. Patterns 3:231-236.
22. Zhao, W. M., A. Seki, and G. Fang. 2006. Cep55, a microtubule-bundling protein, associates with centralspindlin to control the midbody integrity and cell abscission during cytokinesis. Mol. Biol. Cell 17:3881-3896.

What is claimed is:

1. A composition comprising a TEX14 peptide, said peptide having sequence comprising SEQ ID NO:2.

2. The composition of claim 1, wherein the TEX14 peptide is not more than 90, 85, 80, 75, 70, 65, 60, 55, 50, 45, 40, 35, 30, 25, 20, 19, 18, 17, 16, 15, 14, 13, 12, 11, 10, 9, 8, or 7 amino acids in length.

3. The composition of claim 1, wherein the peptide sequence other than SEQ ID NO:2 of the TEX14 peptide comprises the corresponding peptide sequence of SEQ ID NO:1.

4. The composition of claim 1, wherein the peptide sequence other than SEQ ID NO:2 of the TEX14 peptide is at least 75%, 80%, 85%, 90%, 95%, 97%, or 99% identical to the corresponding peptide sequence of SEQ ID NO:1

5. The composition of claim 1, further defined as including a pharmaceutical carrier.

6. A method for inhibiting the proliferation of a mammalian cell, comprising contacting the cell with the peptide of claim 1.

7. The method of claim 6, wherein the mammalian cell is a cancer cell of an individual.

8. The method of claim 7, wherein the cancer cell is a cancer cell of the lung, breast, prostate, pancreas, brain, blood, liver, colon, gall bladder, pituitary gland, spleen, esophagus, ovary, testis, cervix, kidney, salivary gland, anus, or thyroid.

9. The method of claim 7, wherein the method further comprises administering an additional cancer therapy to the individual.

10. The method of claim 9, wherein the additional cancer therapy comprises surgery, radiation, chemotherapy, immunotherapy, or hormone therapy.
